(19) United States
(12) Patent Application Publication
(10) Pub. No.: US 2026/0035535 A1
(43) Pub. Date: Feb. 5, 2026

(54) METHOD FOR DEGRADING POLYCARBONATE

(71) Applicant: NAN YA PLASTICS CORPORATION, TAIPEI (TW)

(72) Inventors: CHING-YAO YUAN, TAIPEI (TW); WEI-SHENG CHENG, TAIPEI (TW); CHAO-TUNG WU, TAIPEI (TW)

(21) Appl. No.: 18/903,109

(22) Filed: Oct. 1, 2024

(30) Foreign Application Priority Data
Aug. 2, 2024 (TW) 113128855

(51) Int. Cl. C08J 11/24; C07C 37/00; C07C 37/82; C07C 37/84; C08J 11/16

(52) U.S. Cl. CPC C08J 11/24; C07C 37/005; C07C 37/82; C07C 37/84; C08J 11/16; C08J 2369/00

(57) ABSTRACT

A method for degrading polycarbonate includes: providing a de-polymerizing solvent; adding a metal hydroxide to the de-polymerizing solvent to form a mixed liquid; adding a polycarbonate material to the mixed liquid to form a reaction liquid, in which the polycarbonate material undergoes a de-polymerization reaction during the de-polymerization operation to form a bisphenol A product; passing the reaction liquid containing the bisphenol A product through an ion exchange column filled with an ion exchange resin to perform ion exchange, thereby forming a purified liquid, in which the metal ions are dissociated from the metal hydroxide; and crystallizing the purified liquid to precipitate bisphenol A crystalline solids from the purified liquid.

### CROSS-REFERENCE TO RELATED PATENT APPLICATION

This application claims the benefit of priority to Taiwan Patent Application No. 113128855, filed on Aug. 2, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a chemical method, and more particularly to a method for degrading polycarbonate.

### BACKGROUND OF THE DISCLOSURE

In the related art, US 2023/0382837 Al (Applicant: LG) and WO 2020/257234 A1 (Applicant: Sabic) disclose methods that use low carbon alcohols (e.g., methanol or ethanol) and co-solvents (e.g., toluene or dichloromethane) to perform de-polymerization reactions on polycarbonate, in which the use of co-solvents favorably enhances the de-polymerization efficiency.

However, residues of the co-solvents may remain in the BPA product, thereby affecting subsequent reactions, and the presence of toxic co-solvents increases the difficulty of subsequent processing. Furthermore, in the related art, the alkali metal catalysts or metal ions that remain in the BPA product after the de-polymerization process can easily lead to quality issues (e.g., poor color tone) in subsequent applications of the BPA product.

### SUMMARY OF THE DISCLOSURE

In response to the above-referenced technical inadequacies, the present disclosure provides a method for degrading polycarbonate.

In order to solve the above-mentioned problems, one of the technical aspects adopted by the present disclosure is to provide a method for degrading polycarbonate, including performing a preparation operation, an addition operation, a de-polymerization operation, an ion exchange operation, and a crystallization purification operation. The preparation operation includes: providing a de-polymerizing solvent, in which the de-polymerizing solvent is a monohydric alcohol solvent. The addition operation includes adding a metal hydroxide to the de-polymerizing solvent to form a mixed liquid. The de-polymerization operation includes: adding a polycarbonate (PC) material to the mixed liquid to form a reaction liquid, in which the polycarbonate material undergoes a de-polymerization reaction during the de-polymerization operation to form a bisphenol A product. The ion exchange operation includes: passing the reaction liquid containing the bisphenol A product through an ion exchange column filled with an ion exchange resin to perform ion exchange for forming a purified liquid, in which the metal ions are dissociated from the metal hydroxide. The crystallization purification operation includes: crystallizing the purified liquid to precipitate bisphenol A crystalline solids from the purified liquid.

In certain embodiments, the monohydric alcohol solvent is phenol, a concentration of the metal hydroxide added is not less than 100 ppm, the de-polymerization operation further includes adding a predetermined amount of water to the mixed liquid to form the reaction liquid, and a water content of the reaction liquid is controlled to be not greater 10 wt %.

In certain embodiments, the preparation operation further includes heating the de-polymerizing solvent to a first heating temperature ranging from 50° C. to 100° C.; in which the de-polymerization operation further includes heating the reaction liquid to a second heating temperature ranging from 110° C. to 150° C.

In certain embodiments, when the water content in the reaction liquid is consumed to be less than 0.5 wt % during the de-polymerization operation, the de-polymerization operation further includes adding water to the reaction liquid to control the water content in the reaction liquid to be between 0.5 wt % and 10 wt %.

In certain embodiments, the ion exchange resin is a strong acid cation exchange resin.

In certain embodiments, the ion exchange resin is a strong acid cation exchange resin with sulfonic acid groups, and a total exchange capacity of the ion exchange resin is not less than 1.0 equivalent per liter.

In certain embodiments, during the ion exchange operation, a metal element content of the reaction liquid is defined as a first metal element content, and the metal element content of the purified liquid is defined as a second metal element content, in which the second metal element content is at least one tenth lower than the first metal element content.

In certain embodiments, the second metal element content is not greater than 10 ppm.

In certain embodiments, during the ion exchange operation, the reaction liquid containing the bisphenol A product, the de-polymerizing solvent, and the metal ions is cooled to an ion exchange temperature before entering the ion exchange column filled with the ion exchange resin, and the ion exchange temperature is between 65° C. and 105° C.

In certain embodiments, an amount of the ion exchange resin in the ion exchange column relative to the reaction liquid is such that every 100 grams of the ion exchange resin is used for ion exchange with 2,000 grams to 3,400 grams of the reaction liquid.

In certain embodiments, the reaction liquid circulates through the ion exchange resin in the ion exchange column, and a circulation flow rate of the reaction liquid is 30 grams to 200 grams per minute.

In certain embodiments, the ion exchange operation further includes analyzing the purified liquid using an Inductively Coupled Plasma Optical Emission Spectrometer (ICP-OES) and controlling the metal element content in the purified liquid to be not greater than 10 ppm.

In certain embodiments, the crystallization purification operation includes: cooling the purified liquid from the ion exchange temperature to a first crystallization temperature ranging from 40° C. to 60° C. under a first reduced pressure maintained between 50 Torr and 150 Torr to precipitate bisphenol A crystalline solids; then, further separating the bisphenol A crystalline solids by filtration and washing the bisphenol A crystalline solids with a washing solvent.

In certain embodiments, the crystallization purification operation further includes: heating the bisphenol A crystalline solids mixed with the washing solvent to a third heating temperature ranging from 105 to 115° C., then cooling to a second crystallization temperature ranging from 40 to 60° C. and maintaining a second reduced pressure between 50 Torr and 150 Torr; then, separating the bisphenol A crystalline solids from the washing solvent by filtration and washing the bisphenol A crystalline solids with a new washing solvent for a second wash; and finally, performing phenol removal under reduced pressure to obtain bisphenol A crystalline products.

Therefore, in the method for degrading polycarbonate by the present disclosure, by virtue of “performing a preparation operation, including: providing a de-polymerizing solvent, in which the de-polymerizing solvent is a monohydric alcohol solvent; performing an addition operation, including: adding a metal hydroxide to the de-polymerizing solvent to form a mixed liquid; and performing a de-polymerization operation, including: adding a polycarbonate (PC) material to the mixed liquid to form a reaction liquid; in which, the polycarbonate material undergoes a de-polymerization reaction during the de-polymerization operation to form a bisphenol A product” and “performing an ion exchange operation, including: passing the reaction liquid containing the bisphenol A product through an ion exchange column filled with an ion exchange resin to perform ion exchange for forming a purified liquid, in which the metal ions are dissociated from the metal hydroxide; and performing a crystallization purification operation, including: crystallizing the purified liquid to precipitate bisphenol A crystalline solids from the purified liquid,” the chemical reaction can be directed to the de-polymerization reaction, thereby enhancing the conversion rate of polycarbonate de-polymerization and increasing the yield of the bisphenol A (BPA) product. The method for degrading polycarbonate provided by the present disclosure effectively avoids the issue of co-solvents (e.g., toluene or dichloromethane) possibly remaining in the product (such as BPA) as encountered in the related art.

Furthermore, the method for degrading polycarbonate provided by the embodiment of the present disclosure can further purify the bisphenol A product through the ion exchange operation and the crystallization purification operation, reducing the residual metal ion content in the bisphenol A product. Accordingly, the purified bisphenol A product is advantageous for subsequent applications, such as effectively improving the color tone of the polymers obtained from the re-polymerization of bisphenol A.

These and other aspects of the present disclosure will become apparent from the following description of the embodiment taken in conjunction with the following drawings and their captions, although variations and modifications therein may be affected without departing from the spirit and scope of the novel concepts of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Method for Degrading Polycarbonate

An embodiment of the present disclosure provides a method for degrading polycarbonate (PC), particularly related to a transesterification method for degrading polycarbonate. The method for degrading polycarbonate of the embodiment of the present disclosure effectively avoids the issue of co-solvents (e.g., toluene or dichloromethane) possibly remaining in the product (e.g., BPA product). Furthermore, the method of the embodiment of the present disclosure can reduce residual metal ions (e.g., sodium or potassium ions) in the product through ion exchange operations and crystallization purification operations, thereby effectively improving the quality of the product. For example, the color tone of a resin produced after re-polymerization of the BPA product can be effectively improved.

Specifically, the method for degrading polycarbonate (PC) of the embodiment of the present disclosure includes step S110, step S120, step S130, step S140, and step S150. It should be noted that the sequence of steps described in the present embodiment can be adjusted according to actual operational needs and is not limited to that which is described in the present embodiment.

Step S110 is to perform a preparation operation, which includes providing a de-polymerizing solvent and adding the de-polymerizing solvent into a reaction tank. The de-polymerizing solvent is a monohydric alcohol solvent. The preparation operation of the present embodiment adopts the monohydric alcohol solvent as the single de-polymerizing solvent.

In some embodiments of the present disclosure, the monohydric alcohol solvent can be at least one selected from the group consisting of: methanol, ethanol, n-propanol, isopropanol, n-butanol, isobutanol, pentanol, hexanol, octanol, isooctanol, nonanol, and phenol. The monohydric alcohol solvent preferably has a boiling point of not less than 100° C. For example, in the present embodiment, the de-polymerizing solvent is phenol. In other words, the preparation operation of the present embodiment adopts phenol as the single de-polymerizing solvent.

In the present embodiment, the preparation operation further includes heating the de-polymerizing solvent up to a first heating temperature. The first heating temperature is between 50° C. and 100° C., preferably between 70° C. and 90° C., and more preferably between 75° C. and 85° C. For example, the first heating temperature is 80° C. in the case of using phenol as the de-polymerizing solvent, but the present disclosure is not limited thereto. The first heating temperature can be appropriately adjusted based on a liquid temperature range of the de-polymerizing solvent.

Step S120 is to perform an addition operation, which includes adding a metal hydroxide into the de-polymerizing solvent in the reaction tank to form a mixed liquid.

In some embodiments of the present disclosure, the metal hydroxide can be at least one selected from the group consisting of alkali metal (Group 1A metal) hydroxides, alkaline earth metal (Group 2A metal) hydroxides, and transition metal hydroxides. For example, the alkali metal hydroxide is sodium hydroxide (NaOH) or potassium hydroxide (KOH). The alkaline earth metal hydroxide is magnesium hydroxide (Mg(OH)₂) or calcium hydroxide (Ca(OH)₂). The transition metal hydroxide is manganese hydroxide (Mn(OH)₂), but the present disclosure is not limited thereto. In the present embodiment, the metal hydroxide is preferably at least one of sodium hydroxide (NaOH) and potassium hydroxide (KOH).

Furthermore, in the mixed liquid, an addition concentration of the metal hydroxide is not less than 100 ppm (parts per million), preferably between 100 ppm and 200,000 ppm, and more preferably between 500 ppm and 10,000 ppm. Specifically, the addition concentration of the metal hydroxide can be between 829 ppm and 8,719 ppm, but the present disclosure is not limited thereto.

Moreover, the addition operation of the embodiment of the present disclosure involves adding an aqueous solution containing the metal hydroxide into the de-polymerizing solvent to mix with the de-polymerizing solvent, thereby forming the mixed liquid that contains the de-polymerizing solvent, the metal hydroxide, and water. The metal hydroxide can be dissociated into hydroxide anion ions (OH⁻) and metal cation ions Mn⁺ (e.g., Na⁺ or K⁺) in the presence of water, exerting a catalytic effect on the subsequent addition of polycarbonate (PC), so as to undergo a de-polymerization reaction.

In the aqueous solution, a weight percentage concentration of the metal hydroxide is between 15 wt % and 60 wt %, preferably between 20 wt % and 45 wt %, and more preferably between 25 wt % and 40 wt %. For example, in one embodiment of the present disclosure, the metal hydroxide added in the aqueous solution is sodium hydroxide (NaOH), and a weight percentage concentration of the metal hydroxide is 32 wt %, but the present disclosure is not limited thereto.

Further, an amount of the aqueous solution containing the metal hydroxide added into the de-polymerizing solvent is approximately between 1/400 and 1/20 of the de-polymerizing solvent, and the weight percentage concentration of the metal hydroxide in the de-polymerizing solvent can be adjusted to be within the above-mentioned concentration range (e.g., not less than 100 ppm) by controlling the amount of the aqueous solution added, but the present disclosure is not limited thereto. The addition operation of the embodiment of the present disclosure can also involve directly adding the powders of the metal hydroxide into the de-polymerizing solvent, and then adding an appropriate amount of water to form a mixed liquid containing a specific concentration of the metal hydroxide.

Step S130 is to perform a de-polymerization operation, which includes: adding a polycarbonate material into the mixed liquid in the reaction tank, and optionally adding a predetermined amount of water into the mixed liquid to form a reaction liquid. Accordingly, the reaction liquid contains: water, the de-polymerizing solvent, the metal hydroxide, and the polycarbonate material.

In the present embodiment, the water content in the reaction liquid is controlled to be not greater than 10 wt %. Furthermore, the polycarbonate material is polycarbonate (PC) granules to be de-polymerized, and the polycarbonate (PC) granules may be formed by crushing recycled polycarbonate waste, but the present disclosure is not limited thereto.

The de-polymerization operation further includes heating the reaction liquid to a second heating temperature, causing the polycarbonate material to undergo a de-polymerization reaction so as to produce bisphenol A (BPA) and carbon dioxide (CO₂) gas.

More specifically, the de-polymerization operation involves heating the reaction liquid from the first heating temperature (e.g., 50° C. to 100° C.) up to the second heating temperature, where the second heating temperature is between 110° C. and 150° C., preferably between 110° C. and 140° C., and more preferably between 110° C. and 135° C. For example, the second heating temperature can be 120° C., but the present disclosure is not limited thereto.

Furthermore, the de-polymerization operation involves continuously stirring the reaction liquid for 1 to 20 hours, preferably 2 to 16 hours, and more preferably 3 to 10 hours after heating the reaction liquid to the second heating temperature, ensuring that the de-polymerization reaction is fully carried out.

It is worth mentioning that in the present embodiment, the de-polymerization operation further includes controlling the water content in the reaction liquid to be between 0.5 wt % and 10 wt %, preferably between 1 wt % and 10 wt %, and more preferably between 1.4 wt % and 9 wt % during the de-polymerization reaction.

Accordingly, the de-polymerization operation can achieve a high de-polymerization conversion rate on the polycarbonate material under the conditions of the metal hydroxide addition concentration (e.g., not less than 100 ppm) and the water content (e.g., 0.5 wt % to 10 wt %).

In an embodiment of the present disclosure, an initial weight ratio of the de-polymerizing solvent relative to the polycarbonate material in the reaction liquid (i.e., an initial weight of the de-polymerizing solvent divided by an initial weight of the polycarbonate material) is between 0.5 and 14, preferably between 0.8 and 5.0, more preferably between 1 and 3.5, and particularly preferably between 1.5 and 3.0.

Further, in the de-polymerization operation (Step S130), the water content in the reaction liquid is controlled to be between 0.5 wt % and 10 wt %. The control method for the water content can, for example, involve extracting a small amount of the reaction liquid from the reaction tank (e.g., 1 ml to 10 ml of the reaction liquid) and detecting the water content in the reaction liquid using a water content detector.

It is worth mentioning that the de-polymerization reaction may decrease the water content in the reaction liquid as the de-polymerization reaction consumes water. The de-polymerization operation of the embodiment of the present disclosure involves monitoring the water content during the de-polymerization reaction. When the water content in the reaction liquid is consumed to be less than 0.5 wt %, the de-polymerization operation can further include adding additional water into the reaction liquid to adjust the water content in the reaction liquid to be between 0.5 wt % and 10 wt %. Accordingly, the de-polymerization operation is beneficial to further degrading the polycarbonate material and reducing the degradation of the bisphenol A (BPA) product, thereby increasing the yield of the bisphenol A product.

It is also worth mentioning that in the de-polymerization reaction, an intermediate product formed from the degradation of the polycarbonate material is diphenyl carbonate (DC), which may be further degraded into phenol (R—OH) and carbon dioxide (CO₂) during the de-polymerization reaction.

Overall, the de-polymerization reaction follows the sequence of Chemical Reaction Mechanism 1 and Chemical Reaction Mechanism 2.

Chemical Reaction Mechanism 1: the polycarbonate (PC) material first undergoes de-polymerization in the presence of a de-polymerizing solvent (i.e., phenol, R—OH, where R is a phenyl group), metal hydroxide (catalyst, M-OH, where M is a metal), and water (H₂O), so as to form bisphenol A (BPA) as a product and diphenyl carbonate (DC) as an intermediate product.

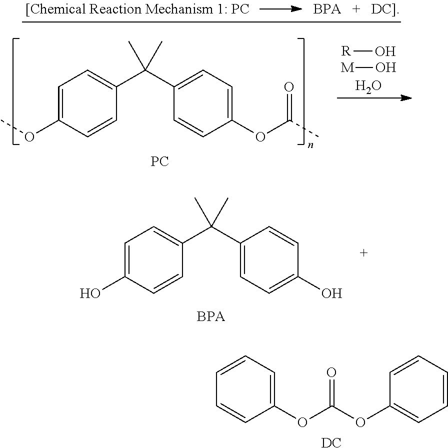

Chemical Reaction Mechanism 2: the diphenyl carbonate intermediate product (DC) is further de-polymerized into phenol (R—OH) and carbon dioxide (CO₂) gas during the de-polymerization reaction in the presence of water.

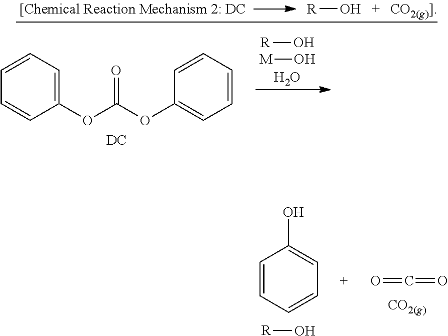

The degradation of the bisphenol A (BPA) product formed from Chemical Reaction Mechanism 1 can be effectively reduced under the aforementioned conditions (i.e., the water content controlled to be between 0.5 wt % and 10 wt %), and only the diphenyl carbonate intermediate product DC will be degraded, thus preserving the bisphenol A product and increasing the yield thereof.

Accordingly, the polycarbonate material is finally formed into a bisphenol A (BPA) product and a phenol (R—OH) byproduct under the aforementioned water content and metal oxide concentration, and generates additional carbon dioxide (CO₂) gas. The degradation conversion rate of the polycarbonate (PC) is 90% to 99% (i.e., the conversion rate of PC from a polymer compound to small-molecule chemicals, representing 90% to 99% of PC being degraded).

It is worth mentioning that since the intermediate product (i.e., diphenyl carbonate, DC) generated from the polycarbonate (PC) material can be further degraded into phenol (R—OH) during the reaction process, and phenol (R—OH) is the same compound as the single de-polymerizing solvent (e.g., phenol) used in the embodiment of the present disclosure, a weight ratio of the de-polymerizing solvent (phenol) relative to the polycarbonate (PC) material will continuously increase during the de-polymerization reaction, thereby facilitating the progress of the de-polymerization reaction and promoting the recovery of the de-polymerizing solvent. Moreover, since the weight ratio of the de-polymerizing solvent relative to the polycarbonate material will continuously increase during the de-polymerization reaction, the initial amount of de-polymerizing solvent that is used can be reduced.

Additionally, it is worth mentioning that, the reaction conditions of the metal hydroxide (e.g., NaOH or KOH) being greater than 100 ppm and the water content being controlled to be 0.5 wt % to 10 wt % are beneficial for the rapid degradation of the polycarbonate (PC) material into bisphenol A (BPA) and carbon dioxide. Accordingly, the polycarbonate degradation reaction can be performed using a single de-polymerizing solvent. If the water content is less than 0.5 wt %, the concentration of the metal hydroxide may become too high (since the metal hydroxide dissociates into hydroxide ions (OH⁻) and metal cations Mn⁺ in water), potentially causing the bisphenol A (BPA) product to degrade in a strong alkaline environment, which is an undesirable reaction. Conversely, if the water content is greater than 10 wt %, the concentration of the metal hydroxide may be too low, thereby reducing the overall de-polymerization efficiency.

Step S140 is to perform an ion exchange operation, which includes: passing the reaction liquid (i.e., crude reaction liquid) formed after the de-polymerization reaction, containing the bisphenol A (BPA) product, the de-polymerizing solvent (e.g., phenol), and metal ions, through an ion exchange column filled with ion exchange resin to perform ion exchange with the metal ions in the reaction liquid, thereby removing the metal ions from the reaction liquid. The metal ions are metal cations dissociated from the metal hydroxide, such as sodium ions (Na⁺).

In the present embodiment, the ion exchange resin is a cation exchange resin, preferably a strong acid cation exchange resin, and more preferably a strong acid cation exchange resin with sulfonic acid groups. The ion exchange operation performs ion exchange and adsorption on the metal ions in the reaction liquid, facilitating the removal of sodium ions or potassium ions from the crude reaction liquid. When the crude reaction liquid passes through the ion exchange resin, the sodium ions or potassium ions in the reaction liquid undergo an acid-base neutralization reaction with the strong acid cation exchange resin, thereby forming salts and water. The salts are adsorbed onto the ion exchange resin, while water, the bisphenol A product, and the de-polymerizing solvent (phenol) can pass through the ion exchange resin and are output from the ion exchange column to form a purified liquid.

The metal element content (e.g., sodium content) in the reaction liquid (e.g., the crude reaction liquid) is defined as a first metal element content, and the metal element content (e.g., sodium content) in the purified liquid is defined as a second metal element content. In the present embodiment, the second metal element content is lower than at least one-tenth of the first metal element content. More specifically, the first metal element content is not less than 100 ppm (corresponding to the aforementioned addition concentration of the metal hydroxide), and the second metal element content is not greater than 10 ppm, preferably not greater than 5 ppm, and more preferably not greater than 2 ppm.

In other words, the metal element content in the purified liquid formed after the ion exchange operation can be effectively reduced, which is beneficial for reducing the metal element content in the bisphenol A crystals obtained after subsequent crystallization, thus facilitating subsequent applications, such as improving the color tone of the high polymers obtained from the re-polymerization of polycarbonate (PC).

It is worth mentioning that in the ion exchange operation, before the reaction liquid (i.e., crude reaction liquid) containing the bisphenol A (BPA) product, the de-polymerizing solvent (e.g., phenol), and the metal ions enters the ion exchange column filled with the ion exchange resin, the reaction liquid needs to be cooled to an ion exchange temperature. In the present embodiment, the ion exchange temperature is between 65° C. and 105° C., and preferably between 80° C. and 105° C.

In other words, the reaction liquid is cooled from the aforementioned second heating temperature between 110° C. and 140° C. to an ion exchange temperature between 65° C. and 105° C.

Accordingly, the reaction liquid is passed through the ion exchange resin in the ion exchange column at an ion exchange temperature between 65° C. and 105° C. to perform ion exchange. The aforementioned temperature range allows the ion exchange resin to have better adsorption on the metal ions (e.g., sodium ions or potassium ions).

If the ion exchange temperature is not cooled to be within the aforementioned range, negative effects may occur. For example, if the ion exchange temperature is too low (e.g., below 60° C.), the bisphenol A (BPA) product and the de-polymerizing solvent (e.g., phenol) may be crystallized, so as to reduce the fluidity of the liquid, which is not conducive to the ion exchange and adsorption operations. In this situation, the bisphenol A product and the de-polymerizing solvent (e.g., phenol) are difficult to pass through the ion exchange resin due to crystallization.

Conversely, if the ion exchange temperature is not low enough (e.g., above 110° C.), the ion exchange temperature may exceed the heat resistance temperature of the ion exchange resin, leading to degradation of the ion exchange resin and causing undesirable substances to precipitate from the ion exchange resin into the reaction liquid, thereby affecting ion exchange effect.

To enhance the efficiency of the ion exchange operation, in some embodiments of the present disclosure, an optimal ratio of the ion exchange resin relative to the reaction liquid (i.e., crude reaction liquid) in the ion exchange column is every 100 grams of the ion exchange resin being used to perform ion exchange and adsorption on 2,000 grams to 3,400 grams of the reaction liquid. That is, the ratio can be, for example, every 200 grams of the ion exchange resin being used to perform ion exchange and adsorption on 4,000 grams to 6,800 grams of the reaction liquid.

Furthermore, the reaction liquid is circulated through the ion exchange resin in the ion exchange column. A circulation flow rate of the reaction liquid is between 30 grams and 200 grams per minute, and preferably between 65 grams and 135 grams per minute. A circulation time of the reaction liquid is between 1 hour and 6 hours, and preferably between 3 hours and 5 hours, so that the ion exchange resin can fully perform ion exchange (acid-base neutralization) and salt (e.g., NaOH) adsorption on the reaction liquid, thereby ultimately outputting the purified liquid through the ion exchange column. During the ion exchange process, the operating temperature of the ion exchange can be controlled at the aforementioned ion exchange temperature (e.g., 65° C. to 105° C., preferably 80° C. to 105° C.).

In one embodiment, the ion exchange operation can further involve analyzing the purified liquid using an Inductively Coupled Plasma Optical Emission Spectrometer (ICP-OES) and controlling the metal element content (i.e., the second metal element content, such as sodium content) in the purified liquid to be not greater than 10 ppm (preferably not greater than 5 ppm, and more preferably not greater than 2 ppm) to ensure the effectiveness of the ion exchange process.

The Inductively Coupled Plasma Optical Emission Spectrometer (ICP-OES) uses high-temperature plasma to excite metal atoms (e.g., sodium atoms) in the sample, and the metal atoms emit light with specific wavelengths as returning to a ground state. By measuring the intensity of these specific wavelengths, the metal atom content in the sample can be quantitatively analyzed. For example, the specific wavelength for sodium atoms is approximately 589 nm. The analysis method can involve sampling 1 gram of the purified liquid, digesting and diluting the sampled purified liquid with nitric acid using microwave digestion, and then analyzing the sodium content using ICP-OES.

Additionally, to enhance the efficiency of ion exchange and adsorption, in one embodiment of the present disclosure, a total exchange capacity of the ion exchange resin can be, for example, not less than 1.0 equivalent per liter (eq/Lt), preferably not less than 1.5 equivalents per liter, and more preferably not less than 1.7 equivalents per liter. The total exchange capacity refers to the amount of ions that can be exchanged by the ion exchange resin per unit volume.

Step S150 is to perform a crystallization purification operation, which includes: crystallizing the purified liquid obtained from the ion exchange operation to precipitate bisphenol A (BPA) crystalline solids from the purified liquid.

More specifically, the crystallization purification operation involves first cooling the purified liquid from the aforementioned ion exchange temperature (e.g., 65° C. to 105° C., preferably 80° C. to 105° C.) to a first crystallization temperature to precipitate the bisphenol A (BPA) crystalline solids. The first crystallization temperature is between 40° C. and 60° C., and preferably between 45° C. and 55° C. The crystallization purification operation involves cooling the purified liquid to the first crystallization temperature under a first reduced pressure, where the first reduced pressure is maintained between 10 Torr (equivalent to millimeters of mercury, mmHg) and 150 Torr, and preferably between 30 Torr and 120 Torr.

Then, the crystallization purification operation further includes separating the bisphenol A (BPA) crystalline solids from the purified liquid by filtration and washing the bisphenol A crystalline solids with a washing solvent (e.g., phenol). A first liquid washing amount of the washing solvent is 8% to 15% by weight of the bisphenol A crystalline solids, and preferably 8% to 12%. Accordingly, the concentration of the metal ions or the metal hydroxides remaining on the bisphenol A crystalline solids can be further reduced.

Subsequently, the crystallization purification operation further includes heating the bisphenol A crystalline solids, which have been mixed with the washing solvent, to a third heating temperature (e.g., 105° C. to 115° C.) and then cooling the bisphenol A crystalline solids to a second crystallization temperature. The second crystallization temperature is between 40° C. and 60° C., and preferably between 45° C. and 55° C. The crystallization purification operation involves cooling the bisphenol A crystalline solids to the second crystallization temperature under a second reduced pressure, where the second reduced pressure is maintained between 10 Torr (equivalent to millimeters of mercury, mmHg) and 150 Torr, and preferably between 30 Torr and 120 Torr.

Thereafter, the crystallization purification operation further includes separating the bisphenol A (BPA) crystalline solids from the washing solvent by filtration and performing a second washing process with a new washing solvent (e.g., phenol) on the bisphenol A crystalline solids. A second liquid washing amount of the new washing solvent is 4% to 8% by weight of the bisphenol A crystalline solids, and preferably 5% to 7%. Accordingly, the concentration of the metal ions (e.g., sodium content) remaining on the bisphenol A crystalline solids can be further reduced.

Finally, the crystallization purification operation includes subjecting the bisphenol A crystalline solids, which have undergone the above two crystallization and filtration washing operations, to phenol removal under reduced pressure to obtain bisphenol A crystals with a purity of not less than 99.5%, and preferably not less than 99.8%. However, the above crystallization purification operation is merely one embodiment of the present disclosure, and the present disclosure is not limited thereto.

Therefore, to address the technical issues in the related art, the present disclosure provides a method for degrading polycarbonate, which includes using a monohydric alcohol solvent (e.g., phenol) as the sole de-polymerizing solvent, allowing the polycarbonate material to undergo degradation in the presence of a metal hydroxide (e.g., NaOH or KOH) at a concentration greater than 100 ppm (preferably 100 to 200,000 ppm, more preferably 500 to 10,000 ppm) and controlling the water content in the reaction liquid between 0.5 wt % and 10 wt % during the de-polymerization reaction. The method of the present disclosure allows the intermediate product (diphenyl carbonate, DC) to be further degraded into phenol (which is the same as the de-polymerizing solvent) and carbon dioxide (which can be removed by venting from the system or by carbon dioxide adsorption), thereby promoting the de-polymerization reaction and increasing the polycarbonate degradation conversion rate to 90% to 99%.

The above method effectively avoids the issue of co-solvents (e.g., toluene or dichloromethane) possibly remaining in the product (e.g., BPA) as seen in the related art.

Furthermore, the method for degrading polycarbonate provided by the embodiment of the present disclosure can further purify the bisphenol A product through the ion exchange operation and the crystallization purification operation to reduce the residual content of metal ion in the bisphenol A product.

Accordingly, the purified bisphenol A product is advantageous for subsequent applications, such as effectively improving the color tone of the polymers obtained from re-polymerization of polycarbonate.

### Experimental Data and Test Results

The content of the present disclosure is detailed in Exemplary Examples 1-1 to 1-3 and Comparative Example 1-1 described below to verify the technical effects of the present disclosure in enhancing de-polymerization conversion rates by controlling the concentration of metal hydroxide and water content in the reaction liquid. However, these examples are merely provided to aid in the understanding of the disclosure and are not meant to limit the scope of the present disclosure.

Exemplary Example 1-1: 500 grams of a de-polymerizing solvent (i.e., phenol) were added into a reaction tank, and a temperature of the de-polymerizing solvent was heated up to 80° C. (i.e., the first heating temperature). 4 grams of a metal hydroxide aqueous solution with 32 wt % NaOH were added into the reaction tank to mix with the de-polymerizing solvent to form a mixed liquid. 200 grams of polycarbonate (PC) granules and 35 grams of water were added into the mixed liquid in the reaction tank to form a reaction liquid. The reaction liquid was heated to 120° C. (i.e., the second heating temperature) and continuously stirred for 5 hours to perform a de-polymerization reaction on the polycarbonate (PC), so as to form bisphenol A (BPA), phenol, and carbon dioxide. A concentration of the metal hydroxide (NaOH) in the reaction liquid was 1,732 ppm, and the water content in the reaction liquid was controlled to be about 4.7 wt %.

Exemplary Example 1-2 and Exemplary Example 1-3 are generally the same as Exemplary Example 1-1 described above, with the main difference being the control of the concentration of metal hydroxide and water content.

Comparative Example 1-1 differs primarily from Exemplary Example 1-1 to Exemplary Example 1-3 in that no additional water was added to the reaction liquid, and the water content in the reaction liquid was 0.18 wt % (uncontrolled), which is lower than the 4.7 wt % in Exemplary Example 1-1 and also below the 0.5 wt % requirement of the present disclosure.

Finally, Exemplary Examples and Comparative example were tested to obtain the de-polymerization conversion rate of polycarbonate (PC) and the yield (%) of the bisphenol A (BPA) product. In the test result items in Table 1 below, the de-polymerization conversion rate of polycarbonate (PC) was tested as follows: First, 10 grams of the crude reaction liquid (i.e., the reaction liquid after the de-polymerization reaction) were taken. The 10 grams of the crude reaction liquid were added to 50 grams of methanol and filtered to obtain a filter cake. The filter cake was washed with 50 grams of methanol. The filter cake was then dried, and the weight of the resulting solid (S) was recorded.

$$De - polymerization conversion rate ( \% ) = ( 100 - S / ( 10 * ( PC / total reaction liquid weight ) ) .$$

In this formula, S is the weight of the solid obtained after drying, 10 grams is the initial weight of the crude reaction liquid that is taken out, PC is the initial amount of polycarbonate (PC) granules, and the total reaction liquid weight is the total weight of the reaction liquid.

Additionally, the yield (%) of the bisphenol A (BPA) product was tested as follows: First, the crude reaction liquid after the de-polymerization reaction was analyzed using High-Performance Liquid Chromatography (HPLC) to determine the concentration of BPA (A). The yield (%) of the bisphenol A (BPA) product was then calculated using the following formula:

$$A / ( PC / total reaction liquid weight ) * 100.$$

In this formula, A is the concentration of BPA, PC is the initial amount of polycarbonate (PC) granules, and the total reaction liquid weight is the total weight of the reaction liquid.

TABLE 1
[Process Conditions and Test Results].

| | | Exemplary | Exemplary | Exemplary | Comparative |
|---|---|---|---|---|---|
| | Items | Example 1-1 | Example 1-2 | Example 1-3 | Example 1-1 |
| Process | Initial amount of | 500 | 500 | 500 | 500 |
| Conditions | depolymerizing | | | | |
| | solvent phenol | | | | |
| | (grams) | | | | |
| | Initial amount of | 4 | 2 | 20 | 4 |
| | metal hydroxide | | | | |
| | aqueous solution | | | | |
| | (32% NaOH) | | | | |
| | (grams) | | | | |
| | Initial amount of | 200 | 200 | 200 | 200 |
| | polycarbonate (PC) | | | | |
| | (grams) | | | | |
| | Initial amount of | 35 | 70 | 14 | — |
| | water (H₂O) | | | | |
| | (grams) | | | | |
| | Concentration of | 1,732 | 829 | 8,719 | 1,818 |
| | metal hydroxide in | | | | |
| | reaction liquid | | | | |
| | (ppm) | | | | |
| | Water content in | 4.7 | 9.0 | 1.4 | 0.18 |
| | reaction liquid | | | | |
| | (wt %) | | | | |
| Test | Depolymerization | 99 | 90 | 99 | 53 |
| Results | conversion rate of | | | | |
| | polycarbonate (PC) | | | | |
| | (%) | | | | |
| | Yield of bisphenol A | 89.3 | 73.2 | 63 | 38 |
| | (BPA) product (%) | | | | |

Table 1 shows that the process conditions in Exemplary Examples 1-1 to 1-3 meet the requirements of the present disclosure for the concentration of the metal hydroxide and the water content controlled in the reaction liquid, the de-polymerization conversion rate of polycarbonate (PC) in each of Exemplary Examples 1-1 to 1-3 is not less than 90%, and the yield of bisphenol A (BPA) is not less than 60%.

Comparative Example 1-1 does not add additional water to the reaction liquid, and the water content in the reaction liquid is 0.18 wt % (uncontrolled), which is lower than the 4.7 wt % in Exemplary Example 1 and below the ideal requirement of 0.5 wt %. The de-polymerization conversion rate of polycarbonate (PC) in Comparative Example 1-1 is 53%, which is significantly lower than the results of Exemplary Examples 1-1 to 1-3.

Additionally, the yield of bisphenol A (BPA) in Comparative Example 1-1 is 38%, which is also significantly lower than the results of Exemplary Examples 1-1 to 1-3.

The following Exemplary Example 2-1 and Comparative Example 2-1 will further verify the impact of ion exchange and crystallization purification operations on the quality of the bisphenol A (BPA) solid crystalline product.

Exemplary Example 2-1: 4,000 grams of de-polymerizing solvent (phenol) were added to a reaction tank, and the temperature of the de-polymerizing solvent was heated to 80° C. (i.e., the first heating temperature). Then, 32 grams of a metal hydroxide aqueous solution (32% NaOH) were added to the reaction tank and mixed with the de-polymerizing solvent to form a mixed liquid. 1,600 grams of polycarbonate granules and 280 grams of water were added to the mixed liquid in the reaction tank to form a crude reaction liquid. The crude reaction liquid was heated to 120° C. (i.e., the second heating temperature) and continuously stirred for 5 hours to perform a de-polymerization reaction, forming bisphenol A (BPA), phenol, and carbon dioxide. The concentration of metal hydroxide (NaOH) in the crude reaction liquid was 1,732 ppm (i.e., sodium content), and the water content in the crude reaction liquid was controlled to be 4.7 wt %.

Subsequently, the crude reaction liquid was cooled to 100° C. (i.e., the ion exchange temperature) and passed through an ion exchange column filled with a strong acid cation exchange resin having sulfonic acid groups to perform an ion exchange operation. The ion exchange operation involved circulating the approximately 5,600 grams of crude reaction liquid (containing 4,000 grams of de-polymerizing solvent and 1,600 grams of polycarbonate degradation product, with water consumed during the de-polymerization reaction) through the ion exchange column filled with 200 grams of the strong acid cation exchange resin having the sulfonic acid groups to perform ion exchange, and after completing the ion exchange operation, outputting a purified liquid. A liquid circulation rate for the ion exchange operation was 100 grams per minute, with a circulation time of 4 hours, to perform acid-base neutralization and adsorption of sodium ions in the crude reaction liquid, thereby forming ion-adsorbed reaction liquid. An ion exchange temperature of the crude reaction liquid was controlled at 100° C. After the ion exchange operation, ICP-OES was used to analyze the sodium content of the purified liquid, and the sodium content was controlled to be less than 2 ppm to confirm the completion of the ion exchange operation. In Exemplary Example 2-1, under the above conditions, the sodium content was approximately 0.32 ppm.

After desorption from the ion resin, the purified liquid was cooled from 100° C. to 50° C. under a reduced pressure (pressure maintained at 100 Torr) to crystallize. The BPA crystalline solid was filtered, and washed with phenol at 10 wt % of the crystalline solid, heated to 110° C., and then cooled to 50° C. under another reduced pressure (pressure maintained at 100 Torr) to crystallize. The BPA crystalline solid was filtered again and washed by second time with phenol at 6 wt % of the crystalline solid, followed by phenol removal under reduced pressure to obtain BPA crystalline solids with a purity greater than 99.8%, and the sodium content in the crystalline solids was undetectable by ICP-OES (result was ND).

Comparative Example 2-1 is generally the same as the de-polymerization operation in Exemplary Example 2-1, with the difference being that Comparative Example 2-1 only proceeded to the end of the de-polymerization operation and did not include subsequent ion exchange and crystallization purification operations. In other words, the sodium content in the crude reaction liquid of Comparative Example 2-1 was 1,340 ppm, indicating that the sodium content in the BPA product far exceeded the 2 ppm requirement.

The BPA product obtained in Exemplary Example 2-1 and the BPA product obtained in Comparative Example 2-1 were further subjected to color tone analysis. The resin color tone in Exemplary Example 2-1 was 5 alpha, while the resin color tone in Comparative Example 2-1 was 232 alpha, which is significantly worse than that in Exemplary Example 2-1.

The color tone analysis method involved dissolving the BPA product in methanol and measuring and analyzing the color tone using a colorimeter to obtain the alpha value of the color tone.

From the above experimental results, it can be seen that, after undergoing ion exchange and crystallization purification following the de-polymerization reaction, the BPA product in Exemplary Example 2-1 significantly reduced the sodium residue in the BPA product and effectively improved the BPA color tone, which is suitable for re-polymerization into polymer resins (PC resins) with better color tone.

### Beneficial Effects of the Embodiments

In conclusion, in the method for degrading polycarbonate by the present disclosure, by virtue of “performing a preparation operation, including: providing a de-polymerizing solvent, in which the de-polymerizing solvent is a monohydric alcohol solvent; performing an addition operation, including: adding a metal hydroxide to the de-polymerizing solvent to form a mixed liquid; and performing a de-polymerization operation, including: adding a polycarbonate (PC) material to the mixed liquid to form a reaction liquid; in which, the polycarbonate material undergoes a de-polymerization reaction during the de-polymerization operation to form a bisphenol A product” and “performing an ion exchange operation, including: passing the reaction liquid containing the bisphenol A product through an ion exchange column filled with an ion exchange resin to perform ion exchange for forming a purified liquid, in which the metal ions are dissociated from the metal hydroxide; and performing a crystallization purification operation, including: crystallizing the purified liquid to precipitate bisphenol A crystalline solids from the purified liquid,” the chemical reaction can be directed to the de-polymerization reaction, thereby enhancing the conversion rate of polycarbonate de-polymerization and increasing the yield of the bisphenol A (BPA) product. The method for degrading polycarbonate provided by the present disclosure effectively avoids the issue of co-solvents (e.g., toluene or dichloromethane) possibly remaining in the product (such as BPA) as encountered in the related art.

Furthermore, the method for degrading polycarbonate provided by the embodiment of the present disclosure can further purify the bisphenol A product through the ion exchange operation and the crystallization purification operation, reducing the residual metal ion content in the bisphenol A product. Accordingly, the purified bisphenol A product is advantageous for subsequent applications, such as effectively improving the color tone of the polymers obtained from the re-polymerization of bisphenol A.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for degrading polycarbonate, comprising:
performing a preparation operation, including: providing a de-polymerizing solvent, wherein the de-polymerizing solvent is a monohydric alcohol solvent;
performing an addition operation, including: adding a metal hydroxide to the de-polymerizing solvent to form a mixed liquid;
performing a de-polymerization operation, including: adding a polycarbonate (PC) material to the mixed liquid to form a reaction liquid; wherein the polycarbonate material undergoes a de-polymerization reaction during the de-polymerization operation to form a bisphenol A product;
performing an ion exchange operation, including: passing the reaction liquid containing the bisphenol A product through an ion exchange column filled with an ion exchange resin to perform ion exchange, thereby forming a purified liquid; wherein the metal ions are dissociated from the metal hydroxide; and
performing a crystallization purification operation, including: crystallizing the purified liquid to precipitate bisphenol A crystalline solids from the purified liquid.

2. The method for degrading polycarbonate according to claim 1, wherein the monohydric alcohol solvent is phenol, a concentration of the metal hydroxide added is not less than 100 ppm, the de-polymerization operation further includes adding a predetermined amount of water to the mixed liquid to form the reaction liquid, and a water content of the reaction liquid is controlled to be not greater 10 wt %.

3. The method for degrading polycarbonate according to claim 2, wherein the preparation operation further includes heating the de-polymerizing solvent to a first heating temperature ranging from 50° C. to 100° C., and wherein the de-polymerization operation further includes heating the reaction liquid to a second heating temperature ranging from 110° C. to 150° C.

4. The method for degrading polycarbonate according to claim 3, wherein, when the water content in the reaction liquid is consumed to be less than 0.5 wt % during the de-polymerization operation, the de-polymerization operation further includes adding water to the reaction liquid to control the water content in the reaction liquid to be between 0.5 wt % and 10 wt %.

5. The method for degrading polycarbonate according to claim 1, wherein the ion exchange resin is a strong acid cation exchange resin.

6. The method for degrading polycarbonate according to claim 1, wherein the ion exchange resin is a strong acid cation exchange resin with sulfonic acid groups, and a total exchange capacity of the ion exchange resin is not less than 1.0 equivalent per liter.

7. The method for degrading polycarbonate according to claim 1, wherein, during the ion exchange operation, a metal element content of the reaction liquid is defined as a first metal element content, and the metal element content of the purified liquid is defined as a second metal element content, and wherein the second metal element content is at least one tenth lower than the first metal element content.

8. The method for degrading polycarbonate according to claim 7, wherein the second metal element content is not greater than 10 ppm.

9. The method for degrading polycarbonate according to claim 1, wherein, during the ion exchange operation, the reaction liquid containing the bisphenol A product, the de-polymerizing solvent, and the metal ions is cooled to an ion exchange temperature before entering the ion exchange column filled with the ion exchange resin, and the ion exchange temperature is between 65° C. and 105° C.

10. The method for degrading polycarbonate according to claim 9, wherein an amount of the ion exchange resin in the ion exchange column relative to the reaction liquid is such that every 100 grams of the ion exchange resin is used for ion exchange with 2,000 grams to 3,400 grams of the reaction liquid.

11. The method for degrading polycarbonate according to claim 9, wherein the reaction liquid circulates through the ion exchange resin in the ion exchange column, and a circulation flow rate of the reaction liquid is 30 grams to 200 grams per minute.

12. The method for degrading polycarbonate according to claim 9, wherein the ion exchange operation further includes analyzing the purified liquid using an Inductively Coupled Plasma Optical Emission Spectrometer (ICP-OES) and controlling the metal element content in the purified liquid to be not greater than 10 ppm.

13. The method for degrading polycarbonate according to claim 1, wherein the crystallization purification operation includes: cooling the purified liquid from the ion exchange temperature to a first crystallization temperature ranging from 40° C. to 60° C. under a first reduced pressure maintained between 50 Torr and 150 Torr to precipitate bisphenol A crystalline solids; then separating the bisphenol A crystalline solids by filtration and washing the bisphenol A crystalline solids with a washing solvent.

14. The method for degrading polycarbonate according to claim 13, wherein the crystallization purification operation further includes heating the bisphenol A crystalline solids mixed with the washing solvent to a third heating temperature ranging from 105 to 115° C., and then cooling to a second crystallization temperature ranging from 40 to 60° C. and maintaining a second reduced pressure between 50 Torr and 150 Torr; then, separating the bisphenol A crystalline solids from the washing solvent by filtration and washing the bisphenol A crystalline solids with a new washing solvent for a second wash; and finally, performing phenol removal under reduced pressure to obtain bisphenol A crystalline products.
